(19) United States
(12) Patent Application Publication
(10) Pub. No.: US 2017/0000530 A1
(43) Pub. Date: Jan. 5, 2017

(54) SURGICAL SPINAL DEVICE

(71) Applicant: THE UNIVERSITY OF NOTTINGHAM, Nottingham (GB)

(72) Inventors: Donal MCNALLY, Nottingham (GB); Bronek BOSZCZYK, Nottingham (GB); Sam EASTERBROOK, Nottingham (GB); John DOMINY, Nottingham (GB); Eyal BEHRBALK, Nottingham (GB)

(21) Appl. No.: 15/104,460

(22) PCT Filed: Dec. 19, 2014
(86) PCT No.: PCT/GB2014/053785

§ 371 Date: Jun. 14, 2016

(30) Foreign Application Priority Data
Dec. 19, 2013 (GB) 1322577.6

(51) Int. Cl. A61B 17/70

(52) U.S. Cl. CPC A61B 17/7079; A61B 2017/0069

(57) ABSTRACT

A spinal manipulation device (30; 80; 90; 100) adapted to protect, in use, a spinal cord from excessive axial and/or shear translations, the spinal manipulation device comprising a guide (31, 32; 81, 82, 83; 91, 92, 93; 101, 102, 103) configured to constrain, in use, movement of a first arm (35; 86; 96;106) relative to a second arm, the first arm and the second arm being fixable to and extending from a segment of spine undergoing manipulation, wherein, in use, the guide constrains movement of the first arm (35; 86; 96; 106) relative to the second arm (36; 87; 97; 107) to be about a substantially fixed centre of rotation located at least partially within the segment of spine undergoing manipulation, thereby protecting the spinal cord from excessive axial and/or shear translations.

The present invention relates to a surgical device, more particularly a spinal manipulation device. In particular, this invention relates to a spinal manipulation device, which is adapted to protect the spinal cord from excessive axial and/or shear translations during surgery. The invention also relates to methods of manufacturing a surgical device and use of the surgical device.

A number of spinal surgical procedures require the manipulation of the spine following removal of one or more parts of the vertebrae. Typically, such a procedure may be carried out in order to correct a deformity such as a kyphotic spine, spondylolisthesis or scoliosis.

Two such procedures are pedicle subtraction osteotomies (PSO) and vertebral column resections (VCR). In a PSO, a wedge is cut in the spine. In a VCR, a whole vertebral body or segment of spinal column is removed.

Before the spine is manipulated, pedicle screws are typically inserted into adjacent vertebrae ready to receive fixation rods at the end of the manipulation. It is common practice to fasten extensions onto these screws to facilitate manipulation.

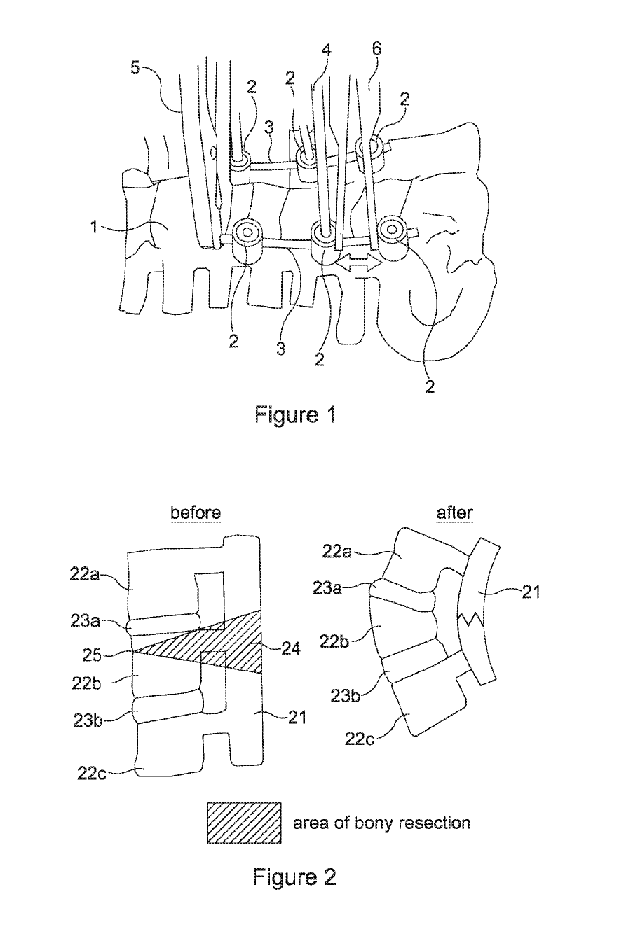

FIG. 1 shows a segment of a patient's spine 1, in which two rows of three polyaxial pedicle screws 2 have been inserted. A rod 3 passes through the heads of each row of three polyaxial pedicle screws 2. A pair of pliers 5 can be used to rotate the rod. An extension 4 is shown fastened to one of the pedicle screws 2. The extension 4 extends substantially perpendicularly from the rod 3 in a generally upwards direction. The extension 4 may be used as a lever to facilitate manipulation. Also shown in FIG. 1, there is a distraction tool 6 for maninpulating the segment.

FIG. 2 illustrates a pedicle substraction osteotomy being performed on a segment of spine 21. The left hand image shows the segment of spine 21 before the pedicle subtraction osteotomy has been performed and the right hand image shows the segment of spine 21 after the pedicle subtraction osteotomy has been performed. The segment of spine 21 contains a plurality of vertebrae 22a, 22b, 22c located one above the other. Each pair of neighbouring vertebrae 22a, 22b, 22c is separated by an intervertebral disc 23a, 23b. As indicated by the hatched area shown in the left hand image, a wedge 24 is cut from the spine, the thick end of the wedge being located posteriorly of the spine and the tip of the wedge being located within the anterior cortex of one of the vertebrae 22b. At the tip of the wedge 24, a hinge 25 is formed within the anterior cortex of the vertebra 22b. The hinge 25 may provide a centre of rotation, when the spine is manipulated. After the wedge has been removed, the spine is manipulated, typically using extension rods attached to pedicle screws (e.g. as shown in FIG. 1 and discussed above), to close the gap created by the resected wedge 24 (see the right hand image in FIG. 2). The pedicle screws and rods may be left in place within the patient to help fix the patient's spine in its new position.

When the spine is being manipulated during a procedure such as a PSO or a VCR, the spinal cord may be unprotected from motions that might shear or stretch it, potentially leading to spinal cord injury.

A spinal surgical procedure such as a pedicle subtraction osteotomy may be relatively risky. For instance, during a spinal surgical procedure such as a pedicle subtraction osteotomy, a patient may be vulnerable to potentially serious spinal cord injury as a result of excessive axial and/or shear translations during surgery.

Whilst being a highly effective corrective method, a PSO may be regarded as an extensive operation with an associated level of risk. Typically, a PSO may require a large section between three vertebral levels to be removed. Following this, the remaining vertebral sections are manipulated through the angle at which the osteotomy wedge was cut and removed. Uncontrolled closure of a PSO can cause a range of problems from minor dural tears and nerve damage to life-threatening accidents such as spinal cord severance and mass haemorrhaging of the patient's aortic vessel.

Eliminating or significantly reducing the risk of an uncontrolled closure of a PSO could greatly improve patient safety and reduce the number of surgical staff required during the procedure.

A first aspect of the invention provides a spinal manipulation device adapted to protect, in use, a spinal cord from excessive axial and/or shear translations, the spinal manipulation device comprising a guide configured to constrain, in use, movement of a first arm relative to a second arm, the first arm and the second arm being fixable to and extending from a segment of spine undergoing manipulation, wherein, in use, the guide constrains movement of the first arm relative to the second arm to be about a substantially fixed centre of rotation located at least partially within the segment of spine undergoing manipulation, thereby protecting the spinal cord from excessive axial and/or shear translations.

In an embodiment, the substantially fixed centre of rotation may be selected such that it is located at the spinal cord or within the anterior cortex of a vertebra.

In an embodiment, the guide may be located between and/or may connect the first arm and the second arm.

In an embodiment, the guide may comprise a pair of smooth curved surfaces, each smooth curved surface having a constant radius of curvature in all curved directions, the smooth curved surfaces being movable, in use, one over the other. The smooth curved surfaces may each be curved in any number of (i.e. one or more) directions. The number of curved directions will determine the directions, in which, in use, the one surface may be moved over the other.

The smooth curved surfaces may each comprise a portion of a cylinder or a portion of a sphere.

In an embodiment, the pair of smooth curved surfaces may be provided by a pair of components which mesh with each other. For instance, the guide may comprise a first component which is shaped and dimensioned to receive, in use, at least a portion of a second component. The first component may have a slot, a groove or a recess, in which, in use, the portion of the second component may be at least partially received. When received by the first component, e.g. located at least in part in the slot, the groove or the recess, the portion of the second component may be movable relative to the first component.

In an embodiment, the smooth curved surfaces may be made from a composite material, which composite material may comprise carbon fibre.

In an embodiment, the guide may comprise a pair of intersecting arcs and a crossover block at the intersection of the arcs configured to allow, in use, movement in a lengthwise direction along both of the arcs. The intersecting arcs may be made from a metal or alloy such as stainless steel or titanium or from a composite material such as a composite material comprising carbon fibre.

In an embodiment, the guide may comprise a universal joint.

In an embodiment, the spinal manipulation device may comprise a clamp or lock operable to prevent movement of the first arm relative to the second arm.

The first arm and/or the second arm may comprise a pedicle screw extension.

The spinal manipulation device may comprise a first arm portion and a second arm portion. In addition, the spinal manipulation device may comprise connecting means for connecting each of the first arm portion and the second arm portion to another element, typically the or a pedicle screw extension. The or each connecting means may comprise a connecting block, e.g. a clamping block.

The spinal manipulation device may be attachable temporarily to pedicle screw extensions.

In an embodiment, the guide may be provided with scale markings.

In an embodiment, at least a portion of the guide may be radiolucent.

The spinal manipulation device may be configured to provide up to a predetermined sagittal correction angle and/or up to a predetermined coronal correction angle. The sagittal correction angle and/or the coronal correction angle may have a wide range of values. In an embodiment, the spinal manipulation device may be configured to provide a sagittal correction angle of up to 60°, up to 50°, up to 45° or up to 40°. Additionally or alternatively, the spinal manipulation device may be configured to provide a coronal correction angle of up to 50°, up to 40°, up to 35° or up to 30°.

In use, the device may sit a distance, e.g. approximately 250 mm, above the pedicle screw heads. Advantageously, this may allow for positioning of radiological equipment to assess the operative site at all times, while also allowing suitable access to the surgical site for the osteotomy process. Restraining the height of the device to below the shoulder height of the surgeon may also limit fatigue during an operative procedure.

In an embodiment, the spinal manipulation device may comprise or be provided with a locating device or positioning instrument.

A second aspect of the invention provides a use of a spinal manipulation device according to the first aspect of the invention.

In order that the invention may be well understood it will now be described by way of example only with reference to the accompanying drawings, in which:

FIG. 1 shows a segment of a patient's spine with pedicle screws and rods inserted therein;

FIG. 2 illustrates schematically a pedicle subtraction osteotomy procedure;

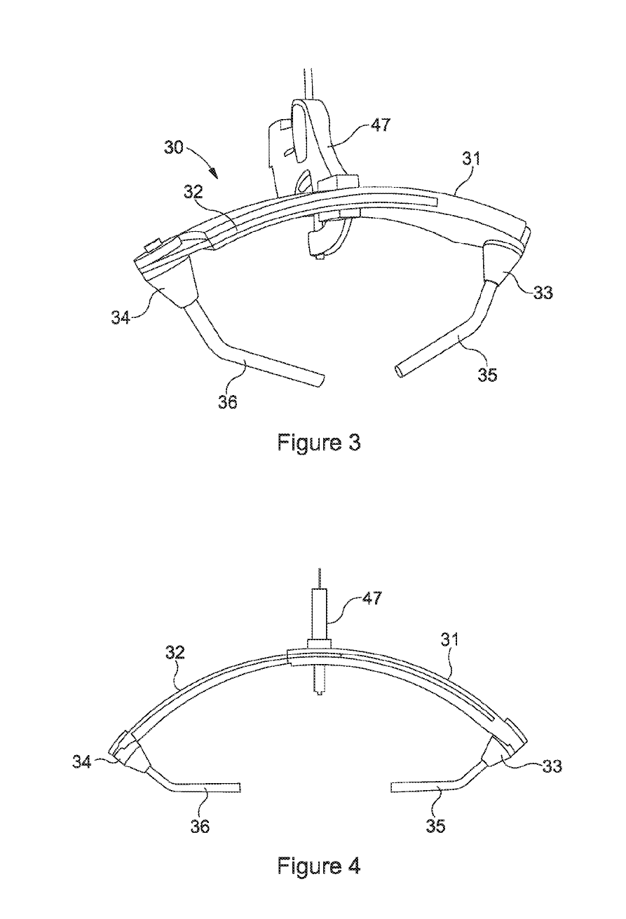

FIG. 3 shows an embodiment of a spinal manipulation device according to the invention in a closed position;

FIG. 4 shows the spinal manipulation device of FIG. 3 in an open position;

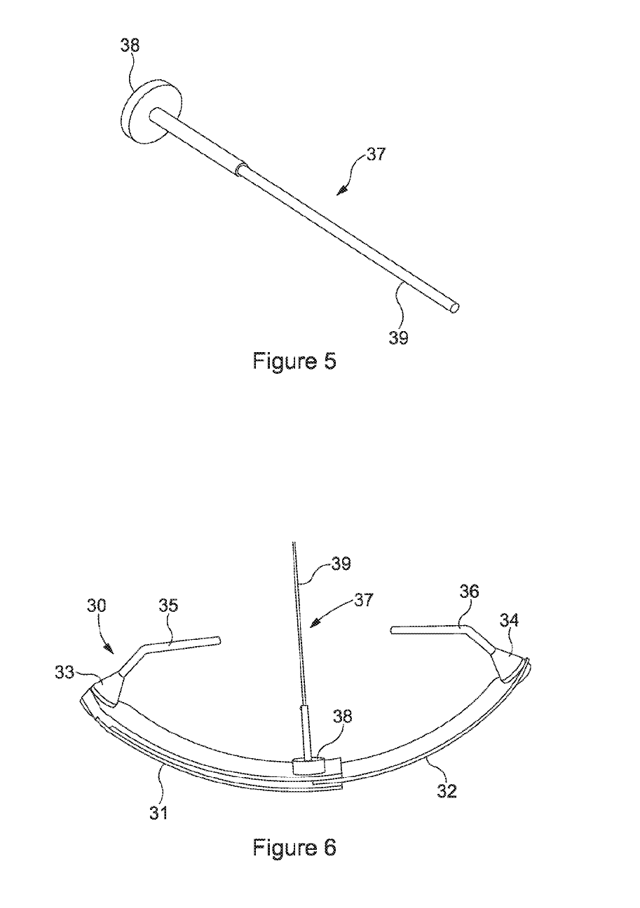

FIG. 5 shows a positioning instrument for use with the spinal manipulation device shown in FIGS. 3 and 4;

FIG. 6 shows the positioning instrument and the spinal manipulation device shown in FIGS. 3 and 4;

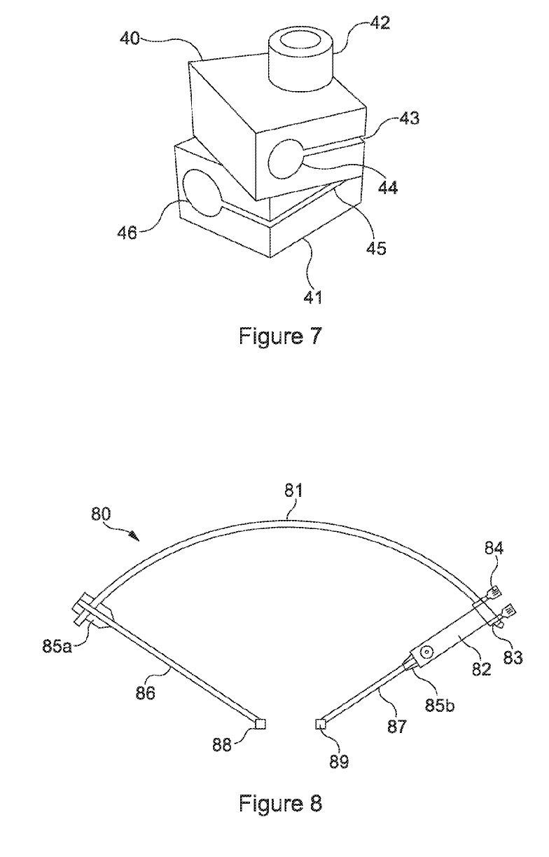

FIG. 7 shows a connecting block for use with the spinal manipulation device shown in FIGS. 3, 4 and 6;

FIG. 8 shows another embodiment of a spinal manipulation device according to the invention;

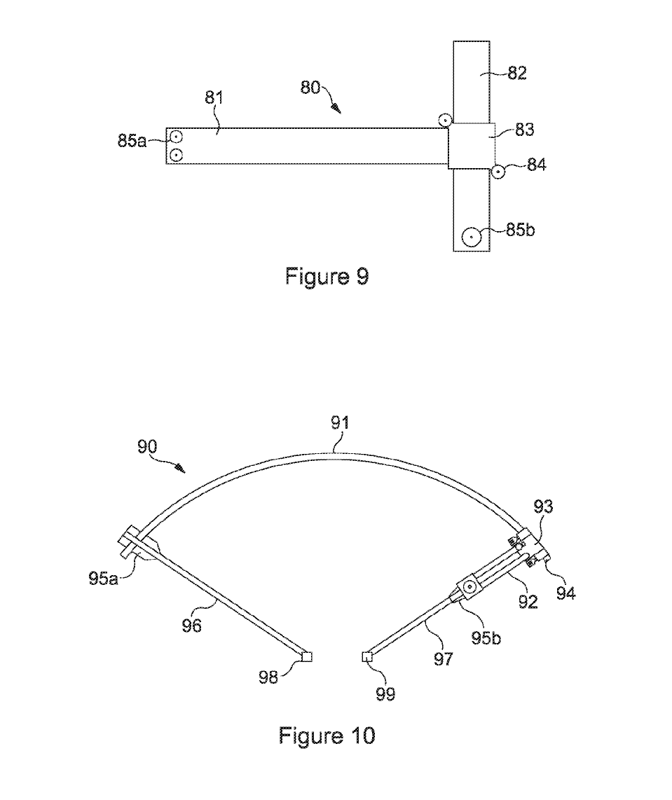

FIG. 9 shows the device shown in FIG. 8 viewed from above;

FIG. 10 shows another embodiment of a spinal manipulation device according to the invention;

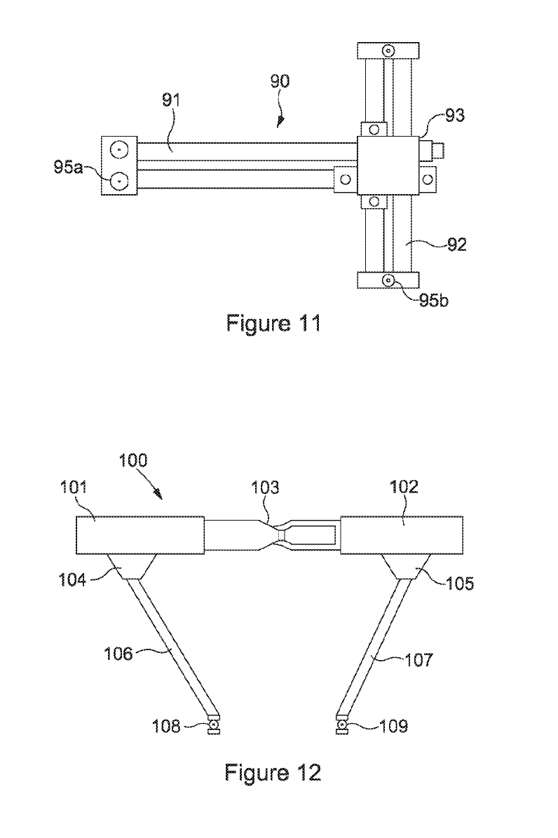

FIG. 11 shows the spinal manipulation device shown in FIG. 10 viewed from above;

FIG. 12 shows another embodiment of a spinal manipulation device according to the invention; and

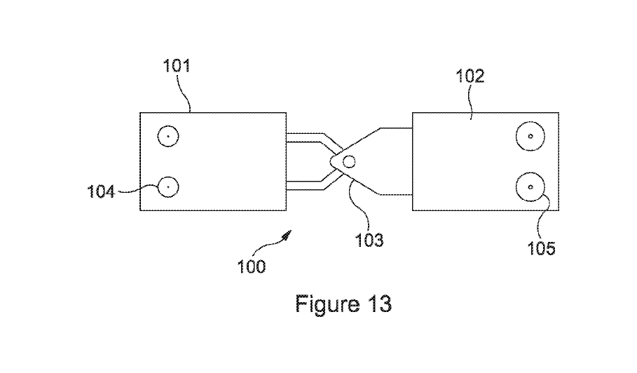

FIG. 13 shows the spinal manipulation device shown in FIG. 12 viewed from above.

Referring to FIGS. 3 and 4, there is shown a spinal manipulation device 30. The spinal manipulation device 30 comprises a guide, which comprises an umbrella structure containing a first composite shell 31 and a second composite shell 32. The second composite shell 32 meshes with the first composite shell 31. The first composite shell 31 and the second composite shell 32 each have the general form of a section of a spherical surface having a substantially constant width along substantially all of its functionally effective length.

The first composite shell 31 comprises an upper sub-shell and a lower sub-shell with a gap between the upper sub-shell and the lower sub-shell. The underside of the upper sub-shell and the topside of the lower sub-shell are both smooth. The second composite shell 32 is received, in use, with minimal tolerance at least partially within the gap between the upper sub-shell and the lower sub-shell.

A first fixation arm 35 and a second fixation arm 36 extend inwardly from either end of the umbrella structure. The first fixation arm 35 is connected to the first composite shell 31. A first, conically shaped, support collar 33 supports the first fixation arm 35 where it is joined to the first composite shell 31. The second fixation arm 36 is connected to the second composite shell 32. A second, conically shaped, support collar 34 supports the second fixation arm 36 where it is joined to the second composite shell 32. The first fixation arm 35 and the second fixation arm 36 comprise a first portion which extends in a substantially radial direction from the first composite shell 31 and the second composite shell 32 respectively. Each fixation arm 35, 36 includes a bend towards the composite shell 31, 32 to which it is connected. The bend is located approximately mid-way along the length of the fixation arm. A second portion of the first fixation arm 35 and a second portion of the second fixation arm 36 extend from the bend to the distal ends of their respective fixation arms.

A clamp 47 is also shown in FIG. 3 and FIG. 4. The clamp is operable to prevent movement of the second composite shell 32 within the gap provided by the first composite shell 31.

FIG. 3 shows the spinal manipulation device 30 in a closed position, and FIG. 4 shows the spinal manipulation device 30 in an open position.

FIG. 5 shows a positioning instrument 37 for use with the spinal manipulation device 30. The positioning instrument 37 comprises a disc-shaped base 38 and a shaft 39 extending from the centre of the base 38. The length of the shaft 39 may be variable. For instance, the shaft 39 may be telescopic.

FIG. 6 shows the positioning instrument 37 and the spinal manipulation device 30. The disc-shaped base 38 of the positioning instrument 37 is in contact with the underside of the lower sub-shell of the first composite shell 31. The shaft 39 points towards the radial centre of the spinal manipulation device 30.

The positioning instrument or locating device can be adjusted to suit the distance of a bony landmark from the desired centre of rotation (e.g. spinal cord). One end is located on the landmark, the other sits anywhere on the underside of the innermost shell.

An alternative to a physical locating device is the use of a crossed laser pointer, line beams. In the case of dots, the separation of the dots (particularly if of different colour) indicates the up or down distance to the intersection point. For example, this could be mounted on the lower shell.

FIG. 7 shows a connecting block for connecting, in use, the spinal manipulation device 30 to a pedicle screw extension connected to a patient's spine. The connecting block contains an upper portion 40 and a lower portion 41. The upper portion 40 and the lower portion 41 are each generally cuboidal in shape. A shaft fitted with a tightening nut 42 connects the upper portion 40 to the lower portion 41. When the tightening nut 42 is loose, the upper portion 40 and the lower portion 41 may be rotated relative to each other about the shaft. When the tightening nut 42 is tightened, the orientation of the upper portion 40 to the lower portion 41 becomes fixed and any objects received in the upper portion 40 and/or the lower portion 41 may be clamped in place.

The upper portion 40 comprises an aperture 44, which extends through the upper portion 40 in a direction perpendicular to the shaft connecting the upper portion 40 to the lower portion 41. A slit 43 extends from the aperture 44 to the edge of the upper portion 40. The shaft connecting the upper portion 40 to the lower portion 41 passes through the slit 43 in a direction perpendicular to the plane of the slit 43. The lower portion 41 comprises an aperture 46, which extends through the lower portion 41 in a direction perpendicular to the shaft connecting the upper portion 40 to the lower portion 41. A slit 45 extends from the aperture 46 to the edge of the lower portion 41. The shaft connecting the upper portion 40 to the lower portion 41 passes through the slit 45 in a direction perpendicular to the plane of the slit 45.

In use, two connecting blocks may connect the fixation arms 35, 36 of the spinal manipulation device 30 to pedicle screws. The fixation arm 35, 36 is received in the aperture 44 in the upper portion 40 of the connecting block and the pedicle screw is received in the aperture 46 in the lower portion 41 of the connecting block or vice versa. The tightening nut 42 is then tightened, thereby clamping the fixation arm and the pedicle screw in place by closing the slits 43, 45 and fixing the orientation of the fixation arm relative to the pedicle screw.

Typically, the composite shells may be manufactured from a carbon fibre composite. Thus, the shells may be very stiff while remaining radiolucent and low mass.

In some embodiments, scales may be added to the composite shells so that the device can be set up and clamped prior to mounting with the expected correction programmed in. During the manipulation, the device may be returned to its closed position and the patient may be automatically aligned as planned. This form of guided manipulation may have applications beyond spinal surgery.

In an embodiment, the device may be configured to accommodate a range of closure angles, typically up to 40° sagittally and/or up to 30° coronally.

The size and dimensions of the spinal manipulation device will depend on its intended use. For instance, to provide 40° of manipulation primarily in the sagittal plane, the arc length of each composite shell may be selected to be around 330 mm. Typically, the required coronal manipulation may be less than the required sagittal manipulation. Thus, for instance, the width of each composite shell may be selected to be around 100 mm.

Each composite shell may have a thickness of around 5 mm.

The spinal manipulation device 30 utilises smooth spherical shells which can glide over one another, thereby twisting and rotating to provide the axis of the rotations around a substantially fixed, typically predetermined, centre of rotation required to close a PSO. Advantageously, the spherical nature of the shells adds rigidity as well as functionality allowing for manipulation in the sagittal, coronal and axial planes of spinal movement.

Typically, the umbrella structure may be made from a high strength composite such as carbon fibre, which may be selected for its forming capabilities, structural properties and radiolucency. The fixation arms and the support collars may be machined out of stainless steel for its compatibility with required sterilisation processes.

Advantageously, the spinal manipulation device 30 may provide for accurate, highly constrained manipulation around a substantially fixed centre of rotation. Typically, in a PSO the substantially fixed centre of rotation may be at the bone hinge, which typically may be located within a vertebra around a third of the way back from the anterior face of the vertebra.

The device should not deflect more than 10 mm at any point during its positioning, operation and manipulation to prevent any damage to spinal or neural structures.

FIGS. 8 and 9 show another embodiment of a spinal manipulation device 80 according to the invention. The spinal manipulation device 80 comprises a first carbon fibre arc 81 and a second carbon fibre arc 82 running perpendicularly to the first carbon fibre arc 81. At the intersection of the carbon fibre arcs 81, 82 there is a crossover block 83, which allows movement along the carbon fibre arcs 81, 82. A locking mechanism 84 is provided on the cross over block 83, which is operable to prevent movement of the carbon fibre arcs 81, 82 relative to each other.

A first fixation arm 86 extends radially inwardly from a first end of the first carbon fibre arc 81. A support collar 85a provides support where the first fixation arm 86 is connected to the first carbon fibre arc 81. A connector 88 for connecting the first fixation arm 86 to a pedicle screw is provided at the distal end of the first fixation arm 86.

A second fixation arm 87 extends radially inwardly from a first end of the second carbon fibre arc 82. A support collar 85b provides support where the second fixation arm 87 is connected to the second carbon fibre arc 82. A connector 89 for connecting the second fixation arm 87 to a pedicle screw is provided at the distal end of the second fixation arm 87.

The composite arcs may be made out of a high grade composite such as carbon fibre. Carbon fibre is suitable from a mechanical and structural perspective and is radiolucent.

Conveniently, the fixation arms may be made out of stainless steel for its structural properties and its compatibility with required sterilisition processes.

In use, the carbon fibre arcs 81, 82 will manipulate a patient's spine in both the sagittal and coronal planes, thereby enabling controlled closure of an osteotomy wedge. The crossover block 83 acts as a guide and the locking mechanism 84 can be used whenever required during an operative procedure to prevent relative movement of the carbon fibre arcs 81, 82. The carbon fibre arcs 81, 82 manipulate the attached vertebrae about the substantially fixed centre of rotation required by the given surgical procedure.

FIGS. 10 and 11 show another embodiment of a spinal manipulation device 90 according to the invention. The spinal manipulation device 90 comprises a first metal arcing arm 91 and a second metal arcing arm 92 running perpendicularly to the first metal arcing arm 91. At the intersection of the metal arcing arms 91, 92 there is a crossover block 93, which allows movement along the metal arcing arms 91, 92. A locking mechanism 94 is provided on the cross over block 93, which is operable to prevent movement of the metal arcing arms 91, 92 relative to each other.

A first fixation arm 96 extends radially inwardly from a first end of the first metal arcing arm 91. A support collar 95a provides support where the first fixation arm 96 is connected to the first metal arcing arm 91. A connector 98 for connecting the first fixation arm 96 to a pedicle screw is provided at the distal end of the first fixation arm 96.

A second fixation arm 97 extends radially inwardly from a first end of the second metal arcing arm 92. A support collar 95b provides support where the second fixation arm 97 is connected to the second metal arcing arm 92. A connector 99 for connecting the second fixation arm 97 to a pedicle screw is provided at the distal end of the second fixation arm 97.

All components of the spinal manipulation device 90 may be made from stainless steel. Stainless steel may be suitable for its mechanical and structural properties and its compatibility with common sterilisation processes for medical devices.

In use, the metal arcing arms 91, 92 will manipulate a patient's spine in both the sagittal and coronal planes, thereby enabling controlled closure of an osteotomy wedge. The carbon fibre arcs 81, 82 manipulate the attached vertebrae about the substantially fixed centre of rotation required by the given surgical procedure. The crossover block 93 acts as a guide and the locking mechanism 94 can be used whenever required during an operative procedure to prevent relative movement of the metal arcing arms 91, 92.

FIGS. 12 and 13 show another embodiment of a spinal manipulation device 100 according to the invention. The spinal manipulation device 100 comprises a first composite body 101 and a second composite body 102. The first composite body 101 is connected to the second composite body 102 by a universal joint 103.

A first fixation arm 106 extends from an underside of the first composite body 101. A support collar 104 provides support where the first fixation arm 106 is connected to the first composite body 101. A connector 108 for connecting the first fixation arm 106 to a pedicle screw is provided at the distal end of the first fixation arm 106. The connector 108 comprises a ball and socket joint allowing for rotation in three axes of rotation during manipulation of the device.

A second fixation arm 107 extends from an underside of the second composite body 102. A support collar 105 provides support where the second fixation arm 107 is connected to the first composite body 102. A connector 109 for connecting the second fixation arm 107 to a pedicle screw is provided at the distal end of the second fixation arm 107. The connector 109 comprises a ball and socket joint allowing for rotation in three axes of rotation during manipulation of the device.

The main functionality of the spinal manipulation device 100 is based around manipulation of the universal joint 103 connecting the first composite body 101 and the second composite body 102. The fixation arms 106, 107 are connected to pedicle screws by connectors 108, 109 comprising ball and socket joints, thereby allowing for rotation in three axes of rotation during manipulation of the device.

The component parts of the spinal manipulation device may be manufactured using any suitable forming technique for the the selected materials. For instance, components made from composite materials such as carbon fibre may be formed using laying up and moulding techniques. Components made from metals such as stainless steel may be formed using machining techniques. Additive and/or subtractive manufacturing techniques may be employed in the manufacture and/or assembly of a spinal manipulation device according to the invention.

By constraining motion of a segment of spine being manipulated to a limited number of, e.g. three, rotations about a substantially fixed centre of rotation, e.g. located at the spinal cord (or anterior cortex in the case of a PSO), a spinal deformity may by corrected without any translation of the spinal cord or injuries to the major blood vessels adjacent the anterior vertebral wall.

The spinal manipulation device may work well if it is attached to polyaxial pedicle screws below the head, so that the head can receive the fixation rod whilst the device is still attached and clamped.

Advantageously, the spinal manipulation device may be capable of being connected to any known system of surgical instrumentation. An example of a system of surgical instrumentation is the Universal Spine System II made be Synthes Spine.

Advantageously, use of the spinal manipulation device according to the invention may prevent any undesired translation of the spinal cord or adjacent neural structures during, for example, the entirety of a PSO procedure on the lumbar section of a patient's spine.

It is envisaged that the spinal manipulation device of the present invention may have a range of potential applications. The different ranges of motion required for each application, in addition to ergonomic considerations, will dictate the size and dimensions of the spinal manipulation device. For example, the spinal manipulation device could be used in: the correction of deformities of a fused or rigid spine using an osteotomy; correction of a kyphosis using posterior instrumentation; scoliosis correction (this could require additional instrumentation).

It may be possible to correct for small translations during the primary angular correction by choice of centre of rotation. For example, a slight anterior displacement of the superior segment (spondylolisthesis) in addition to a kyphosis can be corrected by siting the centre of rotation below the index level.

While the invention has been described mainly with reference to pedicle subtraction osteotomies, it may also have applicability to other spinal surgical procedures, e.g. vertebral column resection. The invention may have applicability to surgery carried out on other sections of the spine and or other parts of the body. The invention may have applicability to surgery carried out on humans or animals.

1-18. (canceled)

19. A spinal manipulation device adapted to protect, in use, a spinal cord from excessive axial and/or shear translations, the spinal manipulation device comprising a guide configured to constrain, in use, movement of a first arm relative to a second arm, the first arm and the second arm being fixable to and extending from a segment of spine undergoing manipulation, wherein, in use, the guide constrains movement of the first arm relative to the second arm to be about a substantially fixed centre of rotation located at least partially within the segment of spine undergoing manipulation, thereby protecting the spinal cord from excessive axial and/or shear translations.

20. The spinal manipulation device according to claim 19, wherein the guide is located between and/or connects the first arm and the second arm.

21. The spinal manipulation device according to claim 19, wherein the guide comprises a pair of smooth curved surfaces, each smooth curved surface having a constant radius of curvature in all curved directions, the smooth curved surfaces being movable, in use, one over the other.

22. The spinal manipulation device according to claim 21, wherein the smooth curved surfaces each comprise a portion of a cylinder or a portion of a sphere.

23. The spinal manipulation device according to claim 21, wherein the pair of smooth curved surfaces is provided by a pair of components which mesh with each other.

24. The spinal manipulation device according to claim 21, wherein the smooth curved surfaces are made from a composite material.

25. The spinal manipulation device according to claim 19, wherein the guide comprises a pair of intersecting arcs and a crossover block at the intersection of the arcs configured to allow, in use, movement in a lengthwise direction along both of the arcs.

26. The spinal manipulation device according to claim 25, wherein the intersecting arcs are made from a metal or alloy such as stainless steel or titanium or from a composite material such as a composite material comprising carbon fibre.

27. The spinal manipulation device according to claim 19, wherein the guide comprises a universal joint.

28. The spinal manipulation device according to claim 19, further comprising a clamp or lock operable to prevent movement of the first arm relative to the second arm.

29. The spinal manipulation device according to claim 19, wherein the first arm and/or the second arm comprises a pedicle screw extension.

30. The spinal manipulation device according to claim 19, wherein the spinal manipulation device comprises a first arm portion and a second arm portion and the spinal manipulation device comprises connecting means for connecting each of the first arm portion and the second arm portion to another element, typically the or a pedicle screw extension.

31. The spinal manipulation device according to claim 19, wherein the guide is provided with scale markings.

32. The spinal manipulation device according to claim 19, wherein at least a portion of the guide is radiolucent.

33. The spinal manipulation device according to claim 19, configured to provide a sagittal correction angle of up to 60°, up to 50°, up to 45° or up to 40°.

34. The spinal manipulation device according to claim 19, configured to provide a coronal correction angle of up to 50°, up to 40°, up to 35° or up to 30°.

35. The spinal manipulation device according to claim 19, which comprises or is provided with a locating device or positioning instrument.
